(19) United States
(12) Patent Application Publication
(10) Pub. No.: US 2019/0247573 A1
(43) Pub. Date: Aug. 15, 2019

(54) METHOD AND SYSTEM FOR SELECTIVE DELIVERY OF A SUBSTANCE TO A TARGETED SURFACE AREA OF THE BODY

(71) Applicant: Richard Postrel, Miami Beach, FL (US)

(72) Inventor: Richard Postrel, Miami Beach, FL (US)

(21) Appl. No.: 16/272,435

(22) Filed: Feb. 11, 2019

Related U.S. Application Data
(60) Provisional application No. 62/629,263, filed on Feb. 12, 2018.

(51) Int. Cl. A61M 5/158; G06F 17/50

(52) U.S. Cl. CPC A61M 5/158; G06F 17/5009; A61M 2205/13; A61M 2005/1588; A61M 2210/06; A61M 2005/1586

(57) ABSTRACT

A medical device and method of use thereof that provides a new means for administering injectable compounds into a targeted area of skin on the body of a human in an automated or semi-automated way, which eliminates the human factors of subjectivity with regards to placement, depth, volume and coverage.

### BACKGROUND

This invention relates generally to a medical device, and in particular to a medical device useful for selectively injecting a substance into targeted areas of the skin of a person in an automated or semi-automated manner.

It is often desirable to be able to deliver via injection a medical, therapeutic, or other type of substance to certain regions or areas of the body of a patient in a targeted manner. For example, cosmetic surgeons will attempt to inject silicone into specific areas of a patient's face in order to achieve a desired result. This process involves manual guesswork as to exactly where the injections should be made. In addition, cosmetic surgeons tend to spread and stretch out the patient's skin during these manual injections, causing damage to the stretched skin.

It is therefore desired to provide a medical device, and method of use of the medical device, that overcomes these and other problems in the prior art.

### SUMMARY

The present invention is a medical device and method of use thereof that provides a new means for administering injectable compounds into a targeted area of skin on the body of a human in an automated or semi-automated way, which eliminates the human factors of subjectivity with regards to placement, depth, volume and coverage.

Thus, this invention provides a device and method of operation of the device that can selectively deliver a substance to a targeted surface region of the body, in particular to the facial or cranial areas. In one embodiment, the device delivers a cannabinoid compound to targeted areas of the scalp in an automated or semi-automated fashion in order to stimulate hair growth in those targeted areas. This provides the means to target hair growth in specified zones in a manner that is far superior to transplants, and without the pain of transplants. In another embodiment, the device delivers a silicone compound or Botox to targeted areas of the face in a semi-automated fashion in order to provide the desired appearance om the face.

In general, the device of the present invention includes an imager for scanning an image of the region of interest of the body, a micro-injector array for injecting the injectable substance to targeted areas of the body, processing and related circuitry for controlling all aspects of the device, a display for displaying the region of interest of the body as well as simulations of how the body will look after injection, user inputs, an optionally a housing tailored to the region of interest of the body in order to simplify the operation of the device, as will be explained in further detail below.

In the first preferred embodiment, the device is used to inject a cannabinoid compound into selected bald areas of the scalp in order to promote hair growth. A human being who experiences baldness, or partial baldness, loses hair from certain, but usually not all, areas of the scalp. Although hair may be absent from certain areas of the scalp, the associated hair follicles within the scalp are still present. These follicles essentially go into hibernation and shut down from further hair growth. Cannabinoid receptors are contained within the hair follicles. If, however, the cannabinoid receptors within these hair follicles are selectively stimulated, hair may once again grow from the follicles.

Thus, in this first embodiment, the array of micro-injectors is configured to inject a cannabinoid compound into targeted areas of the patient's scalp in order to stimulate hair growth in those targeted areas. In particular, each micro-injector injects a controlled amount of the cannabinoid compound into the targeted hair follicles in order to stimulate the targeted cannabinoid receptor and promote the growth of hair from those follicles. Other types of compounds may be injected as well.

The device as described above also includes a headpiece similar in form to a helmet that may be placed over a patient's head, the headpiece containing the imager module and the array of micro-injectors. A container of the substance that is desired to be selectively injected into the patient's scalp is interconnected with the micro-injector array.

This first embodiment operates as follows. The patient will be seated (for comfort), and his or her head will be stabilized during the process, for example by a neck brace to help the patient remain still. The headpiece will be lowered or otherwise placed in close proximity to the scalp of the patient. The imaging process will then occur, in which an image of the entire area of the patient's scalp is obtained. This image data is input to the processing means in order to generate a map of the scalp which will indicate which areas have hair and which do not. The map of the scalp is then processed to determine which of the array of micro-injectors will be activated for injection of the cannabinoid compound. That is, it is desired to inject the compound only into those areas that are devoid of hair.

Next, the micro-injector array is lowered or otherwise caused to make close contact with the patient's scalp. The cannabinoid compound will then be selectively delivered to only those targeted areas of the scalp that do not have hair present. Once the targeted compound delivery is complete, the micro-injector array and headpiece is removed from the patient's head, and the treatment is complete.

Optionally, the display (e.g. a touchscreen display monitor) may be used in order to display the plotting of the intended follicle stimulation via injection. After the initial injection map is generated, a simulation is run and displayed on the display in order to show the patient exactly how the new hair patterns will appear once the hair starts to grow in. The patient and/or technician may then interact with the touchscreen display to modify the map so that hair is grown in differing regions of the scalp. This also allows for fine-tuning of the injection map as desired. Once the patient is satisfied with the simulation, the map is finalized and the process resumes with the injections as described.

In the second embodiment, the invention is used in a cosmetic surgery environment in order to selectively deliver silicone in a targeted manner to the face or other areas of the body. In this embodiment, the patient may provide a photograph of his or her face from a prior time, when the patient was younger. This will be imaged and used as a standard by which the micro-injection procedure will attempt to match. Next, the patient may lie down and the face is imaged and then displayed to the patient and technician/doctor. A comparison is made between the imaged photograph (the younger image of the patient) and the current rendering.

An algorithm is executed by the processing means that determines which areas of the face should be targeted by the micro-injection procedure in order to cause the current facial image to match the one in the photograph.

Next, the micro-injector array is lowered or otherwise caused to make close contact with the patient's face. The silicone compound will then be selectively delivered to only those targeted areas of the face as indicated by the matching algorithm. Once the targeted compound delivery is complete, the micro-injector array is removed from the patient's face, and the treatment is complete.

Optionally, the image of the patient's face from the photograph is displayed on the touchscreen, so the patient may make modifications if desired. That is, desired areas of injection on the face are selected by the user on the touchscreen display. After the initial injection map is generated, a simulation is run and displayed on the monitor in order to show the patient exactly how the injections will manifest on the face. The patient and/or technician may then interact with the touchscreen display to modify the map in order to fine tune the results. Once the patient is satisfied with the simulation, the map is finalized.

Key benefits of the present invention include better dispersal of the treatment, whereby a sequence of injections may be made in order to reduce stress in a given region. This is referred to as feathering or modulating the treatments, which provides the benefits of a more natural and even look. This would not be possible using a manual procedure as in the prior art.

### BRIEF DESCRIPTION OF THE DRAWING

FIG. 1A is a block diagram of a preferred embodiment of the medical device.

FIG. 1B is a block diagram of the device of FIG. 1A used in injecting a cannabinoid compound into the patient's scalp.

FIG. 2 is a flowchart of the operation of the preferred embodiment of the invention.

FIG. 3 is an illustration of the micro-injector array used in the preferred embodiments.

FIG. 4 illustrates a view of a typical micro-injector array from the needle tip end.

### DETAILED DESCRIPTION OF THE PREFERRED EMBODIMENT

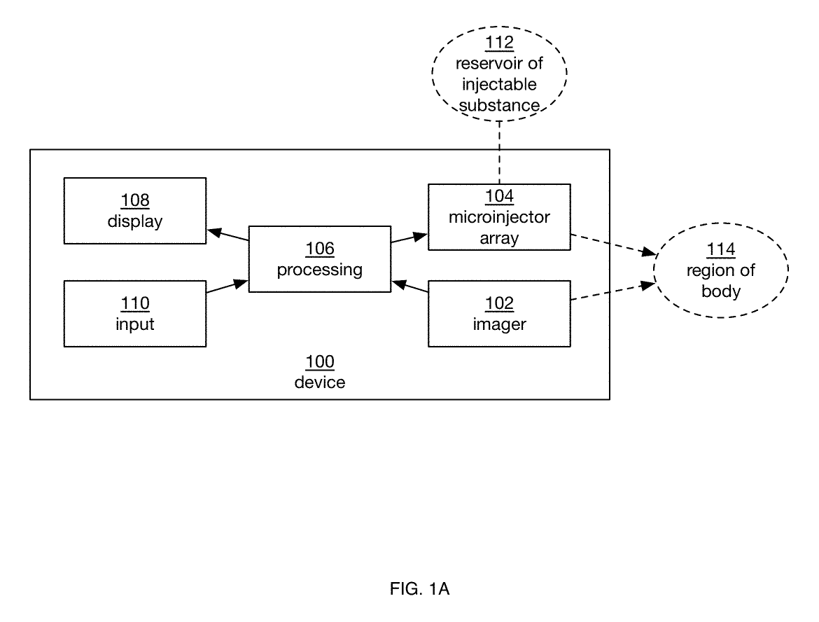

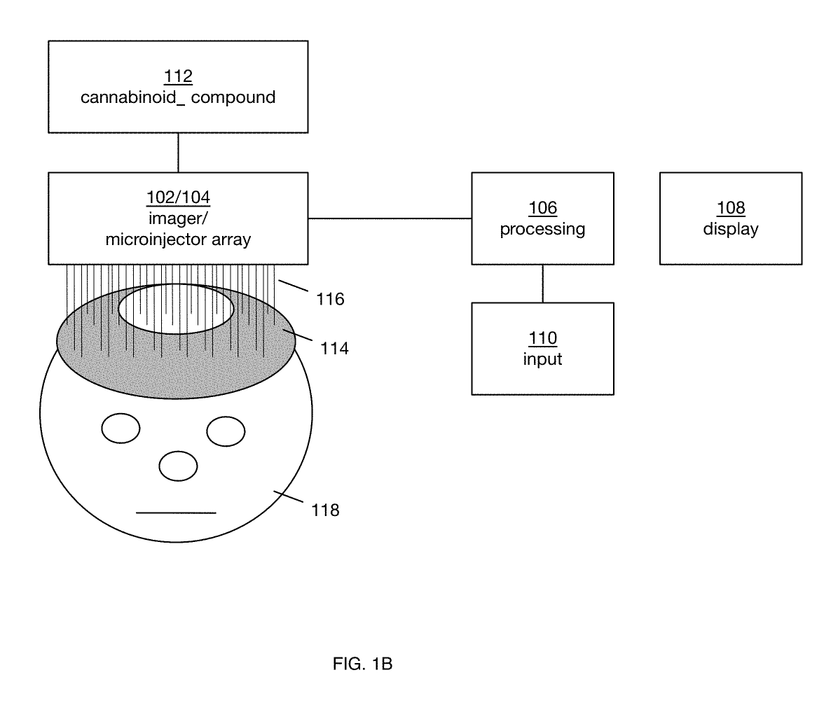

FIG. 1A is a general block diagram of a preferred embodiment of the medical device 100. An imager 102 is provided to acquire a region of the area of interest on the patient. This may be a CCD or CMOS imager similar one found in a digital camera. Optionally, an array of imagers 102 may be provided, for example when a three dimensional part of the body 114 such as the scalp needs to imaged, as will be described above. A micro-injector array 104 is provided for injecting the injectable substance to targeted areas of the body. This includes a large number (e.g. hundreds or even thousands) of micro-injectors or micro-needles that are addressable in order to activate the desired ones when desired. A reservoir 112 of the material suitable for injection is in close proximity to the device for feeding the substance to the micro-injector array 104 so that the micro-injectors may selectively deliver the compound when desired. Processing circuitry 106 is connected to imager 102 and micro-injector array 104, for receiving imaging data, generating the injection map, displaying the injection map, receiving revisions to the injection map, and controlling the micro-injector array 104 for dispensing the compound, as well as other functions, is provided. The processing means 106 may be in a computer that may be a standalone device, or it may be embedded within or in close proximity to the imager 102 and micro-injector array 104. Also shown is a display 108, such as a touchscreen display, that allows for interactively displaying the target region of the body and allowing a user to modify the injection map as may be desired using the touchscreen or an input device 110 such as a pen, mouse or the like.

FIG. 1B is a block diagram of the first preferred embodiment of the device 100, useful for delivering a cannabinoid compound to targeted areas of the scalp 114 in an automated or semi-automated fashion in order to stimulate hair growth in those targeted areas. The modules described above with respect to FIG. 1A are repeated and/or adapted as described. The imager 102 and the micro-injector array 104 are located within a headpiece (not shown) that may be similar in form to a helmet or the like, suitable for placing on the patient's head 118. A reservoir 112 of the injectable substance of cannabinoid compound (or other material suitable for stimulating hair growth as known in the art) is provided in conjunction with the micro-injector array 104 so that the micro-injectors 116 may selectively deliver the compound when desired. The processing means 106 is connected to the imager 102 and the micro-injector array 104, for receiving imaging data, generating the injection map, displaying the injection map, receiving revisions to the injection map, and controlling the micro-injector array for dispensing the compound, as well as other functions. The display 108 and input 11 are provided outside the helmet so the patient and/or doctor may use them interactively as will be described.

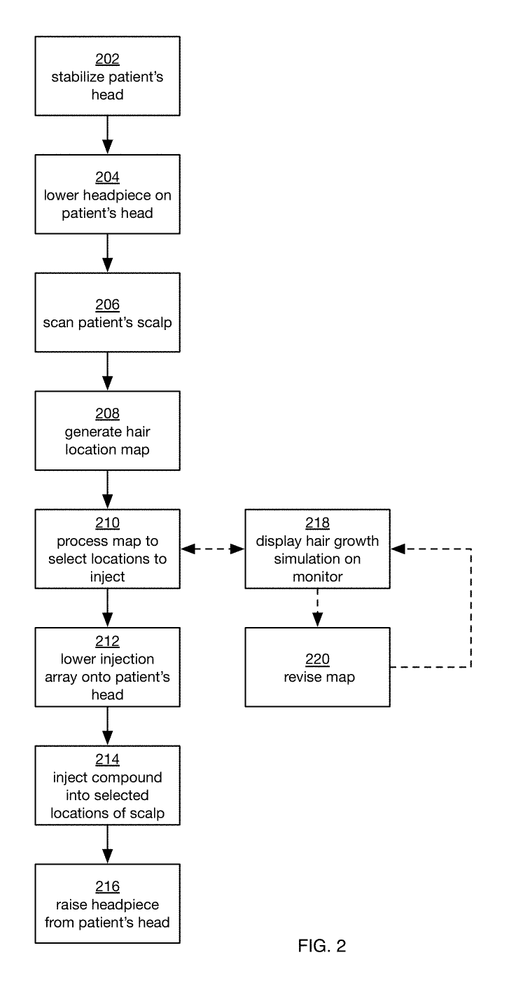

The process of this first embodiment operates generally as shown in FIG. 2. The patient will sit in a comfortable position so that his head may be stabilized at step 202, such as with a neck brace or support. It is important for the patient to have his head remain as stationary as possible in order to ensure the accuracy of the procedure. Once the patient's head has been stabilized, then at step 204 the headpiece the comprises the imager 102 and micro-injector array 1024 is lowered or otherwise placed over the top of the patient's head 118. At step 206, the imager 102 will scan the scalp of the patient. As explained above, the imager 102 may be formed with one or more sensors such as CCD or CMOS sensors, such as those used in modern digital cameras. The sensors would be placed in juxtaposition with each other so as to form a contiguous sensing area that encompasses the scalp of the patient. It is desired to overlap the scalp to ensure that the image includes all of the areas of the head in which hair exists or would be desired to be grown. For example, in FIG. 1B, the scalp area imaged by the array 102 includes the area 114 where hair is in sufficient supply as well as where the patient is bald, shown in the middle of the scalp in this example. It may be desired for the patient to have his existing hair trimmed to a very short length so as to enable the imaging array 102 to obtain an accurate scan of the scalp.

After the scalp has been imaged, then at step 208 that data is input to the processing means 106, which analyzes the image data provided by the imager 102, and then generates a map of the scalp where hair is and is not located. At step 210, the map is processed in order to select the locations that need to be stimulated for growth with the injectable compound. Since the intent here is to only apply the cannabinoid compound in those desired areas where the hair does not exist, it is important to filter out the areas where hair already exists when generating the map.

The doctor or technician operating the device will input various parameters to generate a treatment algorithm, for example the depth of penetration of each micro-injector, the amount

Docket No.: 560-004RP of compound being injected at a particular micro-injector, the duration of each micro-injection, and the like. This is also referred to as automated micro-needling. The treatment algorithm may also provide for an injection pattern to yield optimal results. For example, it may be desired to activate all targeted micro-injectors at the same time, or it may be desired to activate certain ones at one time and then other ones that are offset at a different time, perhaps milliseconds apart. Micro-injections may be made on a repetitive basis in small amounts, rather than all at once. The dosage may be spread out over a region using nearby micro-injectors if desired. Different dosage amounts may be delivered by different micro-injectors, thus delivering different amounts of the compound to different targeted areas of the scalp, as may be desired. In addition, there may be a correlation between dosage density, tensile strength, and the like, that determine the quantity of the compound being injected as well as the density of the injections.

Telemetry may be used whereby the addressable micro-injectors are positioned on the head relative to specific facial or cranial biomarkers. For example, at least three points may be selected on the patient by which all subsequent locations for injections are derived from an analysis of those points.

The optional step 218, where the patient and/or technician may modify the map manually, may now be invoked. At step 218, the scalp image data and injection map that has been generated at step 210 are used to generate a display of a simulation of how the hair growth will appear. The display 108 may be a touchscreen display that will allow the patient or technician to interact and modify the injection map at step 220 so that the hair growth projection will also change in real time. This will allow the patient to fine-tune the injection parameters so as to obtain the exact look he or she would like. Once this iterative process has completed, the process returns to step 210.

At step 212, the array of micro-injectors that have been selected to inject the cannabinoid compound into the patient's scalp are lowered and placed into close proximity to the patient's scalp. At step 214, the compound is injected through the selected injectors into the scalp, and at step 216 the headpiece is raised or otherwise removed.

In the second preferred embodiment, the device 100 is used in a cosmetic surgery environment in order to selectively deliver silicone or the like in a targeted manner to the face or other areas of the body. If the patient desires the treatment to restore him to look like he did in his youth, then he may provide a photograph of his face from that prior time. This will be scanned or otherwise input and used as a standard by which the micro-injection procedure will attempt to match. Next, the patient lies down and the face is imaged by the imaging array 102 and then displayed to the patient and technician/doctor on the display 108.

The processor 106 will then analyze the scanned photograph and the acquired scanned image of the patient, and perform an algorithm that determines how the micro-injections should occur in order to restore the patient as closely as possible to the image from the photograph. Optionally or in addition, the desired areas of injection on the face are selected by the user on the display 108. After the initial injection map is generated, a simulation is run on the processor 106 and displayed on the display 108 in order to show the patient exactly how the injections will manifest on the face. The patient and/or technician may then continue to interact with the display 108 to modify the map in order to fine tune the results. Once the patient is satisfied with the simulation, the map is finalized.

Next, the micro-injector array 104 is lowered or otherwise caused to make close contact with the patient's face. The silicone compound will then be selectively delivered to only those targeted areas of the face that are indicated in the map. Once the targeted compound delivery is complete, the micro-injector array 104 is removed from the patient, and the treatment is complete.

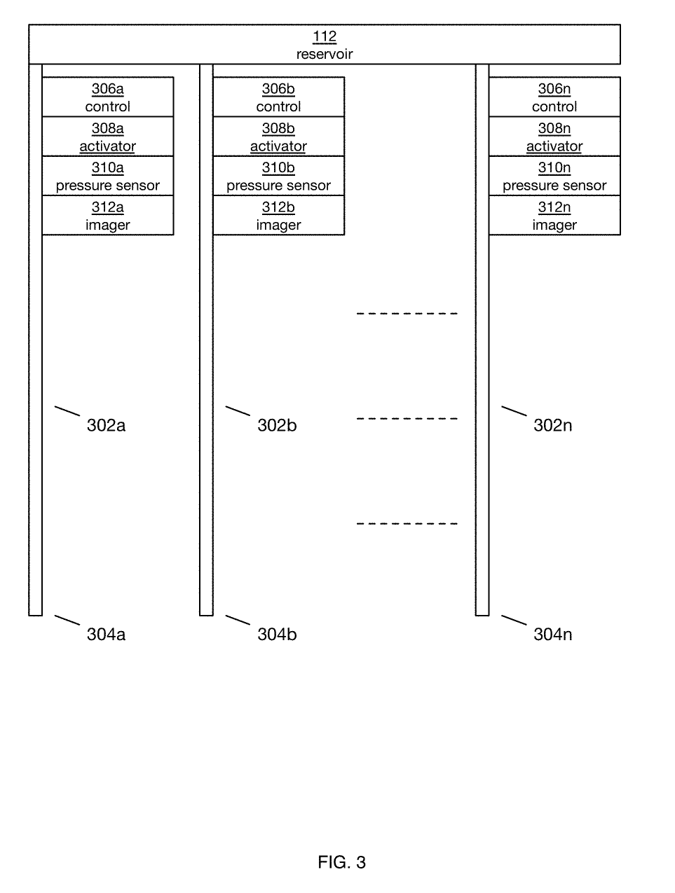

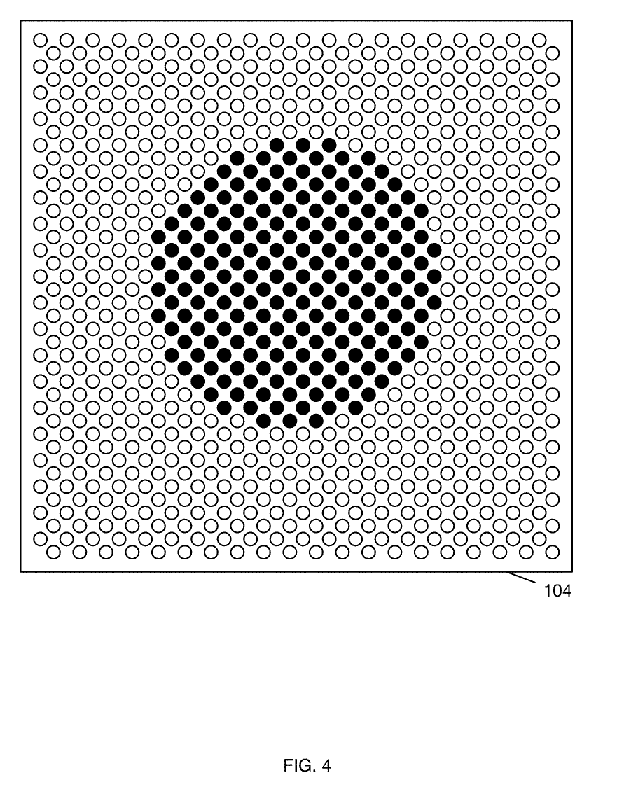

The construction and operation of the micro-injector array 104 will now be described with respect to FIG. 3 and FIG. 4. The micro-injector array 104 is comprised of a multiplicity of micro-injectors 302 (also referred to as micro-needles). Shown in FIG. 3 are micro-injectors 302a, 302b, . . . 302n, wherein n is the total number of micro-injectors in the entire array. Only three micro-injectors 302 are shown, but it is understood that the multiplicity of micro-injectors will be implemented in a two-dimensional array as desired by the system designer. That is, the number of micro-injectors 302, placement, area of coverage, etc. will vary according to the specific application being implemented.

Each micro-injector 302 comprises a micro-needle 304, a control 306, an activator 308, a pressure sensor 310, and an imager 312. Additionally, each of the micro-injectors 302 is interconnected with the reservoir 112, which will hold the compound being injected into the patient.

The control 306 is an electronically addressable controller that enables the processor 106 to select the micro-injectors 302 in the array 104 that should be activated for a given treatment. Reference is now made to FIG. 4, which illustrates a view of a typical array 104 from the needle tip end. Each circle in the array represents an individual micro-injector 302, wherein the darkened tips graphically illustrate the specific micro-injectors 302 that have been selected by the processor 106 to deliver the compound to the patient in a given treatment, and the non-darkened tips indicate the specific micro-injectors 302 that have not been selected by the processor 106 to deliver the compound to the patient. Each of the micro-injectors 302 are selected via their address in the array 104 as known in the art.

The activator 308 is a mechanical activator, such as but not limited to a hydraulic device, that will cause the micro-injector 302 to move towards the patient's skin in order to deliver the compound from the reservoir when that micro-injector 302 is selected for delivery, and to raise the micro-injector 302 away from the patient's skin when delivery has been completed. The pressure sensor 310 operates to detect the pressure of the micro-needle 302 against the patient's skin, feed that pressure information to the processor 106 so that the processor may regulate the operation of the activator 308 and adjust the pressure of the micro-injector 302 against the patient's skin as may be desired for a given treatment.

The imager 312 provides an image of the target area of the patient to the processor 106 so it may aid in calculating the injection parameters (pressure, speed, duration) that are used by the processor in controlling the operation of each micro-injector 302. Optionally, the collection of imagers 312 may be used to perform the functions of the imager 102 (i.e. collect an initial image of the area to be treated such as the scalp). Or, as described above, a separate imager array may be used to perform this function.

In an alternative embodiment, multiple compound reservoirs 112 may be utilized. A micro-injector may be programmed to be obtain the injectable compound from any of the available reservoirs. This enables multi-therapy whereby different compounds can be injected through different injectors so that they work either independently or in collaboration with one another.

What is claimed is:

1. A process comprising
lowering a headpiece onto a head of a patient,
scanning the scalp of the patient,
generating a hair location map,
processing the map to select locations to inject a compound,
lowering an injection array onto the head of the patient,
injecting the compound into selected locations of the scalp, and
raising the headpiece from the head of the patient.

2. The process of claim 2 further comprising
displaying a hair growth simulation on a monitor, and
revising the map.
